(19) United States
(12) Patent Application Publication
(10) Pub. No.: US 2007/0209083 A1
(43) Pub. Date: Sep. 6, 2007

(54) Cell and transgenic animal modelling human antigenic presentation and their uses

(75) Inventors: Kader Thiam, Lyon (FR); Frederique Rattis, Chapel Hill, NC (US); Fabien Bertaux, Grezieu La Varenne (FR); Alexandre Fraichard, Versailles (FR)

Correspondence Address: BUCHANAN, INGERSOLL & ROONEY PC, POST OFFICE BOX 1404, ALEXANDRIA, VA, 22313-1404, US

(73) Assignee: GENOWAY, Lyon (FR)

(21) Appl. No.: 11/709,838

(22) Filed: Feb. 23, 2007

Related U.S. Application Data
(63) Continuation of application No. 10/483,569, filed on Oct. 28, 2004, now abandoned.

(30) Foreign Application Priority Data
Jul. 13, 2001 (FR) 01/09352

(51) Int. Cl. A01K 67/027; C12N 5/06; C12N 15/09

(52) U.S. Cl. 800/14; 800/18; 435/354; 435/325; 800/15; 800/16; 800/17; 435/455

(57) ABSTRACT

The invention concerns an isolated animal cell comprising at least a transgene including at least a nucleotide sequence coding for at least a human polypeptide involved in the recognition and/or antigenic activation by T cells. The invention is characterised in that said cell, or a progeny of said cell, expresses at least all or part of the or said human polypeptide(s), and the homologous endogenous animal gene coding for an animal polypeptide homologous with said human peptide is invalid. The invention also concerns the corresponding transgenic animal. The cell and the transgenic animal of the invention can be used in a method for screening compounds which modulate an immune response in humans. The invention further concerns the use of the inventive cell as cell rendered autologous or tolerated by the immune system.

This application is a continuation of application Ser. No. 10/483,569 filed Oct. 28, 2004, which is the national stage of PCT application No. FR02/20475 filed Jul. 12, 2002, which claims priority from French Application No. 01/09352 filed Jul. 13, 2001.

This invention relates to the domain of biology and more particularly the domain of animal transgenesis and immunology. The invention relates to an isolated animal cell comprising at least one transgene comprising at least one nucleotide sequence encoding at least one human polypeptide involved in antigenic recognition and/or cell activation of T cells, characterised in that said cell, or a progeny of said cell expresses at least whole or part of the human polypeptide(s), and characterised in that the homologous endogenous animal gene encoding an animal polypeptide homologous to said human polypeptide is invalid. The invention also relates to the corresponding transgenic animal. The cell and the transgenic animal according to the invention may be used in a process for screening compounds that modulate an immune response in humans. The invention also relates to the use of the cell according to the invention as an autologous cell or as a cell tolerated by the immune system for preparation of a medicine for the treatment of patients requiring a cell and/or tissue graft.

Recognition of an antigen by T cells involves a tripartite complex composed of molecules of the Major Histocompatibility Complex (MHC) located on the surface of Antigen Presenting Cells (APC), the antigen peptide and the T cell receptor (TCR). Thus, to achieve T lymphocyte activation, the peptide must be correctly prepared by the APCs and then associated with molecules of the major histocompatibility complex (MHC) (denoted H-2 in the mouse, HLA in humans) and finally expressed at the surface of APCs, so that the presented peptide-MHC complex can be recognised by the specific TCR.

MHC molecules are composed of two α and β chains. Each of these chains can be coded by different alleles existing on the short arm of the chromosome 6 (6p21.3) in humans. Loci encoding genes of class II molecules are centromeric and are located in the HLA-D region (about 900 kb). HLA-A contains at least 20 class II genes, of which 9 are functional (DPB1, DPA1, DQB1, DQB2, DRB1, DRB2, DRB4, DRB5, DRA). The class I region (about 1600 kb) contains about 20 class I genes, of which 8 have been named officially in a precise nomenclature (A, B, C, D, E, F, G, H, J); only products A, B, C were studied in detail. Structurally, class I MHC molecules are formed from an a chain non-covalently associated with a polypeptide, beta 2 microglobulin (β2m).

The HLA polymorphism represents variations within a locus in a population. Each variation represents an HLA allele. For example, the association of an a chain with a chain enables the expression of a functional class II MHC protein, with a peptide binding site at which most polymorphous variations will be concentrated.

This phenomenon, defined as genic restriction, influences peptide-MHC interactions and partly explains why some individuals respond and others do not respond to a given antigen. Animal models (mice) were used initially to study the nature of the immune response set up (for example Th1 versus Th2, humoral versus cell-mediated response, etc.). However, they have shown their limits, in particular for studies searching for a “vaccinal candidate” that could be extrapolated to human. Transgenic mice expressing a given allele of the MHC were used to partly circumvent the genic restriction of MHC. In most cases, these models were obtained by conventional transgenesis. This means that the gene encoding the HLA molecule is randomly integrated into the mouse genome, since the effect of the integration site on the biological activity of the transgene cannot be ignored. Furthermore, these initial models obtained by conventional transgenesis done for wild mice, express endogenous murine MHC molecules in addition to the human MHC molecule. The immune response observed in response to a vaccination protocol then reflects the presentation of the antigen by the combination of these two types of MHC molecules (human and murine). One alternative to this problem was to cross transgenic mice with mice with a targeted deactivation of murine MHC genes (MHC I: Pascolo et al., 1997; MHC II: Ito et al., 1996).

These models, that only take account of a given HLA, are very reducing since a single individual naturally expresses different HLAs. Furthermore, these models are not relevant since the transgene encoding human HLA is integrated into the genome in a random manner, such an integration necessarily having consequences on the regulation and expression of endogenous genes of the integration site and on the precise regulation of the expression of the transgene. Thus, there is a real need for multi-transgenic animals (mice or other) for several human class I and/or class II MHC molecules described as being the most representative of a given population; these animals would constitute tools with a considerable value for a preliminary evaluation of the capacity of some molecules to initiate an immune response in humans. This is the technical problem that this invention is intended to solve.

In order to circumvent the above-mentioned restrictions, the inventors propose to introduce several human HLA alleles into the genome of laboratory animals, preferably in the mouse, and thus cover a wide range of the human genic restriction related to MHC. Preferably, multigenic HLA mouse models developed by the inventors express one to two class I and/or class II HLA molecules. Indeed, association with a class I HLA molecule and one to two class II HLA molecules in the same model would be a valuable tool for vaccinology studies. Indeed, MHC class I molecules will have peptides exogenous to CD8+ T lymphocytes, responsible for a CTL type response (cytotoxic T lymphocytes). Class II HLA molecules will present peptides to CD4+ T lymphocytes which, after their activation, will produce cytokins and thus enable development of a cell-mediated and/or humoral immune response. In having a transgenic animal for human class I and class II molecules, the two components necessary for studying an immune response will be met, and the model obtained will be useful for studying an antigen (restricted class I) in association with a peptide (restricted class II) that facilitates development of a global T response. Despite the fact that all the steps preceding the antigenic presentation (preparation of the antigen, etc;) remain fully “murinised”, such a double transgenic model will be significantly more relevant biologically.

The inventors propose to eliminate the expression of murine MHCs to only allow the introduced human HLA genes to be expressed, thus increasing the quality of the model. For example, the targeted insertion (Knock-In) technology is used for this purpose and eliminates the disadvantages of random insertion of the transgene obtained by conventional transgenesis by microinjection of DNA, for example into the pronucleus. Thus, murine MHC molecules will be invalidated at the same time as the human HLA molecules are introduced. These human genes replacing their murine equivalents, benefit from the endogenous regulation normally acting on expression of MHC molecules during development of an immune response.

The inventors propose to use this same targeted insertion technique to humanise the β2m molecule to eliminate possibilities of association between human class I HLA and murine β2m molecules. The presentation of restricted antigen to class I MHC molecules will then be as close as possible to that observed in human cells.

Finally, still with the objective of increasing the relevance of the model as a function of these potential applications, the inventors propose to humanise, in addition to MHC molecules, other molecules playing an important role in the recognition of antigens such as CD4 and CD8 co-receptors. It has been demonstrated that CD4 and CD8 molecules combine with the TCR-MHC-peptide complex in the form of a quaternary complex. This association does not take place between xenogenic molecules under satisfactory conditions, as was demonstrated in the earlier models of transgenic mice carrying a human HLA incapable of interacting with murine CD4 (Barzaga-Gilbert et al., 1992). The CD4 and CD8 molecules engaged jointly with the TCR in the bond of MHC-peptide complexes can stimulate intracellular signals essential in the lymphocytic activation process. This is why the invention also relates to an HLA multi-transgenic mouse model in which a chimeric gene is introduced into the corresponding CD4 and CDB murine locus by targeted insertion, this chimeric gene preferably encoding the extracellular part of the human CD4 or CD8 molecule and the transmembrane and intracellular part of the murine molecule; therefore the MHC/CD4 or CD8 recognition in such an animal model is human, while the transduction of the signal within the T lymphocyte is murine.

Therefore, this invention is intended to provide humanised HLA multi-transgenic animal models, and preferably mice models, for all molecules playing a key role in the initiation of an immune response, while preserving signalling in the murine T lymphocyte. Therefore, the purpose of the invention is to supply a collection of HLA multi-transgenic laboratory animals in different genetic pools that will all be experimental models for preliminary evaluation of molecules of interest (antigens or others). The evaluation thus made will be very relevant to the extent that the antigen will be presented in an optimally humanised context.

The models according to the invention form refined models useful for the study of antigenic tolerance (induction or rupture), vaccinology, allergic and/or inflammatory phenomena (delayed hypersensitivity). HLA multi-transgenic animals according to the invention can also be used to reproduce experimental auto-immune pathology models described in humans and associated with one or several given HLAs: for example by expressing HLAs that are observed with an unbalanced bond in populations and are associated with auto-immune pathology phenotypes.

More specifically, the invention is related to an isolated animal cell comprising at least one transgene comprising at least one nucleotide sequence encoding at least one human polypeptide involved in antigenic recognition and/or in cell activation of T cells, characterised in that said cell, or a progeny of said cell, expresses all or at least part of said human polypeptide(s), and characterised in that said nucleotide sequence is integrated into the genome of said cell in a stable manner by a targeted insertion by homologous recombination (Knock-in) at least one, preferably two alleles of said endogenous animal gene, the integration of said sequence invalidating said homologous endogenous animal gene.

For the purpose of this invention, a homologous polypeptide refers to polypeptides from different animal species, one being human, optionally with a substantial sequence homology and encoding functionally equivalent polypeptides in the two animal species.

A human polypeptide involved in antigenic recognition and/or cell activation of T cells refers to all molecules involved in antigenic recognition and/or cell activation of T lymphocytes.

Antigenic recognition refers to presentation of the antigen to T cells by an MHC molecule leading to activation of said T cells, and therefore initiation and development of an immune response.

Cell activation of T lymphocytes refers to the entire response cascade induced after priming of the immune or pathological response.

The human polypeptide involved in the recognition and/or antigenic activation by T cells is selected in the group composed of the antigens of the major histocompatibility complex (HLA), of the β2-microglobulin, T cell receptor (TCR) chains, polypeptides of the CD3 complex, CD4 and CD8 co-receptors, the co-stimulating molecules ICAM-1, ICAM-2, ICAM-3, LFA-1, CD28, CD80, CD86, CD40, CD40L, CD5, CD72, CTLA-4, CD2 and LFA-3. More precisely, said antigen of the major histocompatibility complex is selected in the group composed of type I, type II and type III HLA antigens.

Preferably, but not limitatively, said human polypeptide is a human class I HLA antigen preferably chosen from among functional human class I HLA antigens, and preferably in the group composed of HLA-A2, HLA-A24, HLA-A1, HLA-A3, HLA-B7, HLA-B27, HLA-B44, HLA-B8, HLA-B35, HLA-CW7, HLA-CW3 and said invalidated homologous animal polypeptide is a MHC I animal antigen that is preferably a functional animal class I MHC molecule. Even more preferably, the animal used is the mouse. The murine antigen of the invalidated class I major histocompatibility complex is therefore chosen as a function of the murine genetic pool. Thus, the H2K and H2D antigens are preferably deactivated in mice from strain 129 or C57/B16, and the H2L antigen is preferably deactivated in Balb/c mice.

Preferably, but not limitatively, said human polypeptide is a human class II HLA antigen preferably chosen from among functional human class II HLA antigens, and even more preferably from the groups composed of HLA-DR4, HLA-DR1, HLA-DR11, HLA-DR7, HLA-DR2, HLA-DR3, HLA-DQ8, HLA-DQ3, HLA-DP4 and said invalidated homologous animal polypeptide is an MHC II animal antigen that is preferably a functional animal class II MHC molecule. Since the animal is preferably an inbred mouse, the murine antigen of the MHC II to be invalidated is chosen as a function of the murine genetic pool; thus, the I-E beta antigen, that is not expressed and therefore is not functional in the murine strain 129, is not chosen when the targeted transgenesis is done in strain 129. Preferably, the I-A alpha, I-A beta and I-E alpha antigens are invalidated in strain 129 mice.

The invention can be made in any mammal cell competent for homologous recombination. Preferably, rodent cells, and particularly mouse, rat, hamster or guinea pig cells will be used. Preferably, mouse cells will be used. Alternatively, cells of primates (including human cells) such as monkeys, chimpanzees, macaques, baboons, may be used. Cells from bovines, caprinae, ovines, porcines, in particular small pigs, equidae such as horses, lagomorphs such as rabbits may be used.

Cells according to the invention may be functionally defined as being capable of achieving homologous recombination of the fragment (s) of exogenous DNA that contains at least one and preferably two regions with sequence homologies with an endogenous cell DNA sequence. These cells naturally contain endogenous recombinases or were genetically modified to contain them or to contain the compounds necessary to realise DNA recombination.

Preferably, among the cells according to the invention, it is worth mentioning all cell types naturally expressing specific proteins involved in recognition and/or antigenic activation by T-cells. Cells in the immune system, professional and non-professional antigen presenting cells, and hematopoietic stem cells should all be mentioned.

Among these cells, it is worth mentioning cells in the immune system, and non-exhaustively mature and immature T lymphocytes, thymocytes, dendritic cells, intra-epithelial lymphocytes, NK cells, B lymphocytes, basophiles, mastocytes, macrophages, eosinophiles, monocytes, platelets, Langerhans cells, dendritic cells, professional and non-professional antigen presenting cells. For example, cells according to the invention may also be neurone cells. It is also worth mentioning cells that under some culture conditions, or after differentiation or genetic modification, are capable of expressing specific proteins involved in recognition and/or antigenic activation by T cells. Hematopoietic stem cells, totipotent (ES cells) or multi-potent embryonic stem cells may also be cited. These stem cells may be differentiated as a cell expressing specific proteins according to the invention. Stem cells mean all types of undifferentiated multipotent or pluripotent cells that can be cultivated in vitro for a prolonged period without losing their characteristics, and that can be differentiated in one of several cell types when they are placed under defined culture conditions. Thus, when the cell according to the invention is an ES cell or an hematopoietic cell, it could be possible to induce differentiation of the cell in different cell types that could express the protein(s) specific to recognition and/or antigenic activation by T cells, for example such as cells in the immune system, and more precisely mastocytes, basophiles, monocytes, eosinophiles, mature and immature T lymphocytes, thymocytes, dendritic cells, NK cells, B lymphocytes, Langerhans cells, platelets, monocytes, dendritic cells, professional and non-professional antigen presenting cells.

When embryonic stem cells (ES) have to be used, for example to produce the transgenic animal according to the invention, a cell line of ES cells may be used or embryonic cells may be obtained freshly from a host animal according to the invention, usually a mouse, a rat, a hamster or a guinea pig. Such cells are cultivated on a layer of appropriate feeder fibroblasts or on gelatine, in the presence of appropriate growth factors such as the Leukaemia Inhibitory Factor (LIF).

More generally, cells according to the invention correspond to all animal cells, preferably mammal cells, except for human cells. Therefore examples of mammal cells competent for recombination comprise fibroblasts, endothelial cells, epithelial cells, cells usually cultivated in laboratory such as Hela cells, CHO (Chinese Hamster Ovary) cells, for example Dorris, AE7, D10.64, DAX, D1.1, CDC25.

For the purposes of this invention, a transgenic cell means a cell containing a transgene. “Transgene” or exogenous nucleic acid sequence or exogenous gene means genetic material that was or will be artificially inserted in the genome of a mammal, particularly in an in vitro cultivated mammal cell or in a living mammal cell, or that will be maintained in said cell in episomal form. Preferably, the transgene according to this invention comprises at least one sequence that could be transcribed or transcribed and translated into a protein. The transgene(s) according to the invention or their expression does (do) not affect operation of the biological network of the immune system, nor more generally operation of the biological network of the cell. The transgene may be cloned in a cloning vector that propagates the transgene in a host cell and/or optionally in an expression vector to express the transgene. The recombinant DNA technologies used for construction of the cloning vector and/or expression vector according to the invention are known and commonly used by persons skilled in the art. Standard techniques are used for cloning, isolation of DNA, amplification and purification; enzyme responses involving ligase DNA, polymerase DNA, restriction endonucleases are made according to the manufacturer's recommendations. These and other techniques are usually used according to Sambrook et al., 1989). Vectors include plasmids, cosmids, phagemids, bacteriophages, retroviruses and other animal viruses, artificial chromosomes such as YAC, BAC, HAC and other similar vectors.

Methods of generating transgenic cells according to the invention are well known to the man skilled in the art (Gordon et al., 1989). Various techniques for transfecting mammal cells have been described (review given in Keon et al., 1990). The transgene according to the invention is optionally included in a linearised or non-linearised vector, or in the form of a vector fragment, and can be introduced into the host cell using standard methods such as for example micro-injection into the nucleus (U.S. Pat. No. 4,873,191), transfection by precipitation with calcium phosphate, lipofection, electroporation (Lo, 1983), thermal shock, transformation with cationic polymers (PEG, polybrene, DEAE-Dextran, etc.) viral infection (Van der Putten et al., 1985), sperm (Lavitrano et al., 1989).

According to one preferred embodiment of the invention, the transgenic cell according to the invention is obtained by gene targeting of the transgene(s) at one or more sequences of the genome of the host cell. More precisely, the transgene is inserted stably by homologous recombination at homologous sequences in the genome of the host cell. When the objective is to obtain a transgenic cell in order to produce a transgenic animal, the host cell is preferably an embryonic stem cell (ES cell) (Thompson et al., 1989).

Gene targeting represents directed modification of a chromosomic locus by homologous recombination with an exogenous DNA sequence with a sequence holomogy with the targeted endogenous sequence. A distinction is made between different types of genetic targeting. Thus, gene targeting may be used to modify, and usually increase, the expression of one or several endogenous gene(s), or to replace one endogenous gene by an exogenous gene, or to place an exogenous gene under the control of elements regulating the gene expression of the particular endogenous gene that remains active. In this case, the gene targeting is called knock-in (KI). Alternatively, gene targeting may be used to reduce or eliminate the expression of one or several genes. This gene targeting is then called knock-out (KO) (see Bolkey et al., 1989).

According to this invention, integration in the genome of said cell of said transgene encoding at least one human polypeptide involved in the recognition and/or antigenic activation by T cells, forms a knock-in; it is done at the level of said endogenous genes encoding a homologous animal or encoding one of said animal polypeptide(s) such that said transgene invalidates expression of said endogenous gene. The cell according to the invention is characterised in that the transgene is stably integrated into the genome of said cell and in that its expression is controlled by regulation elements of the endogenous gene. Stable integration means insertion of the transgene in the genomic DNA of the cell according to the invention. The transgene thus inserted is then transmitted to cell progeny. The transgene is integrated in the upstream side, the downstream side or in the middle of the target endogenous gene. According to one preferred embodiment, the cell according to the invention expresses one or several transgenes, each encoding at least one human polypeptide involved in the antigenic recognition and/or cell activation of T cells.

In order to achieve this homologous recombination, the transgene must contain at least one DNA sequence comprising all or at least part of the gene encoding the human polypeptide involved in the antigenic recognition and/or activation of T cells, possibly with the required genetic modifications and optionally one or several positive or negative selection genes, and also homology DNA regions homologous with the target locus, preferably two regions, located on each side of the portion of the reporter gene. “Homology DNA regions” or “homologous or substantially homologous DNA sequences” means two DNA sequences which, after optimal alignment and after comparison, are identical for at least about 75% of nucleotides, at least about 80% of nucleotides, normally at least about 90% to 95% of nucleotides, and even better at least about 98 to 99.5% of nucleotides. “Identity percentage” between two sequences of nucleic acids for the purposes of this invention refers to a percentage of nucleotides identical in the two sequences to be compared obtained after the best alignment, this percentage being purely statistical and the differences between the two sequences being randomly distributed over their entire length. “Best alignment” or “optimum alignment” means the alignment for which the identity percentage determined as described below is the highest. Comparisons of sequences between two nucleic acid sequences are traditionally made by comparing these sequences after aligning them optimally, the said comparison being made by segment or by “comparison window” to identify and compare local regions for similar sequences. For the comparison, sequences may be optimally aligned manually, and also using the Smith and Waterman local homology algorithm (1981), the Neddleman and Wunsch local homology algorithm (1970), the Pearson and Lipman similarity search method (1988), and computer software using these algorithms (GAP, BESTFIT, BLAST P, BLAST N, FASTA and TFASTA in the Wisconsin Genetics Software Package, Genetics Computer Group, 575 Science Dr., Madison, Wis.). Preferably, the optimum alignment is obtained using the BLAST program with the BLOSUM 62 matrix. The PAM or PAM250 matrices can also be used. The identity percentage between two sequences of nucleic acids is determined by comparing these two optimally aligned sequences, the sequence of nucleic acids or amino acids to be compared possibly including additions or deletions from the reference sequence for optimal alignment between these two sequences. The identity percentage is calculated by determining the number of identical positions for which the nucleotide or the amino acid residue is identical between the two sequences, by dividing this number of identical positions by the total number of compared positions and multiplying the result obtained by 100 to obtain the identity percentage between these two sequences. The expression “nucleic sequences with an identity percentage of at least 85%, preferably at least 90%, 95%, 98% and 99% after optimum alignment with a reference sequence” refers to nucleic sequences with some modifications from the reference nucleic sequence, particularly such as a deletion, a truncation, an elongation, a chimeric fusion, and/or a substitution, particularly an isolated substitution, and for which the nucleic sequence has at least 85% and preferably at least 90%, 95%, 98% and 99% identity after optimum alignment with the reference nucleic sequence. The length of homology regions is partially dependent on the degree of homology. This is due to the fact that a reduction in the homology quantity results in a reduction in the homologous recombination frequency. If non-homologous regions exist in different portions of homologous sequences, it is preferable if this non-homology does not extend over the entire portion of the homologous sequence, but is restricted to discrete portions. In all cases, the weaker the degree of homology, the longer the homology region needs to be to facilitate the homologous recombination. Although as little as 14 pb 100% homologous is sufficient to make the homologous recombination in bacteria, in general longer homologous sequence portions are preferred in mammal cells. These portions are at least 250 pb, 500 pb, 750 pb, 1000 pb, 1500 pb, 1750 pb, 2000 pb, 2500 pb, 3000 pb, 4000 pb and preferably at least 5 000 pb for each homologous sequence portion. According to the invention, DNA fragments may be of any size. The required minimum size depends on the need to have at least one homology region sufficiently long to facilitate homologous recombination. The size of DNA fragments is equal to at least about 2 kb, and preferably at least about 3 kb, 5 kb and 6 kb.

The transgene is not limited to a particular DNA sequence. Thus, homologous DNA sequences present in the transgene may have a purely synthetic origin (for example routinely made starting by a DNA synthesiser), or they may be derived from mRNA sequences by reverse transcription, or they may be directly derived from genomic DNA sequences. When the homology DNA sequence is derived from RNA sequences by reverse transcription, it may or may not contain all or part of non-coding sequences such as introns, depending on whether or not the corresponding RNA molecule has been partially or totally spliced. Preferably, homologous DNA sequences used to make the homologous recombination include genomic DNA sequences rather than cDNA. Indeed, important cis-regulating sequences present in introns, distal regions, promoting regions may be present. Sequences derived from genomic DNA usually encode at least a portion of a gene but alternatively may encode untranscribed regions or regions in an unrearranged genetic locus such as loci of immunoglobulins or T cell receptor. In general, genomic DNA sequences include a sequence encoding an RNA transcript. Preferably, the RNA transcript encodes all or part of a polypeptide; preferably, they are human polypeptides involved in antigenic recognition and/or cell activation of T cells. When the transgene encodes part of the polypeptide, it is preferably one or several exons; thus within the context of humanisation of the murine gene of beta-2-microglobulin, the transgene used preferably comprises an exon; in this case, the knock-in is preferably an exchange of exons. Alternatively, the same transgene can encode several human genes. In this case, human genes are preferably in the form of cDNA and are placed under the control of human promoting regions. When several human genes are thus bound contiguously, they are either arranged in the form of multiple distinct gene entities, each comprising at least one promoter, regulating sequences, a coding sequence, termination signals, or the coding sequences are dispersed in CIS, separated by Internal Ribosomal Entry Sites (IRES) and are placed under the control of the same group of transcription and translation regulation sequences.

Preferably, IRESs are selected from among IRESs of the encephalomyocarditis virus (EMCV), the cardiovirus, the aphtovirus, the enterovirus, the rhinovirus, in particular human rhinovirus (HCV), the hepatitis A virus, the type I poliovirus, the foot and mouth disease virus (FMDV), the ECHO virus, the murine leukaemia virus (MLV) of cMyc.

According to one preferred embodiment, the transgene comprises at least one nucleotide sequence encoding all or at least part of a human polypeptide involved in antigenic recognition and/or cell activation of T cells, a positive selection cassette that may or may not be surrounded by sites specific to the action of recombinases, for example a Lox/Neo-TK/Lox or Lox/Neo/10× or FRT/Neo-TK/FRT or FRT/Neo/FRT cassette possibly also being in a position 5′ of the said nucleotide sequence, and characterised in that a negative selection cassette containing for example the DTA and/or TK genes is present at least one of the ends of the transgene.

The transgene may be as small as a few hundred pairs of cDNA bases, or as large as about a hundred thousand pairs of bases of a genic locus comprising the exonic-intron encoding sequence and regulation sequences necessary to obtain an expression controlled in space and time. Preferably, the size of the recombined DNA segment is between 2.5 kb and 1 000 kb. In any case, recombined DNA segments can be smaller than 2.5 kb and longer than 1 000 kb.

The transgene of this invention is preferably in native form, in other words is derived directly from an exogenous DNA sequence naturally present in an animal cell. This DNA sequence in native form may be modified, for example by insertion of restriction sites necessary for cloning and/or insertion of site-specific recombination sites (lox and flp sequences). Alternately, the transgene of this invention may have been created artificially in vitro by recombining DNA techniques, for example by associating genomic DNA and/or cDNA segments. This is chimeric transgene. The DNA sequence according to the invention, in native or chimeric form, may be mutated using techniques well known to the man skilled in the art. For coding sequences, these mutations may affect the amino acid sequence.

When the cells have been transformed by the transgene, they may be cultivated in vitro or they may be used to produce transgenic animals. After transformation, the cells are seeded on a feeder layer and/or on in an appropriate medium. The cells containing the construction may be detected using a selective medium. After being left for long enough to allow colonies to grow, the colonies are retrieved and analysed to determine if a homologous recombination and/or integration of the construction occurred. Positive and negative markers, also called selection genes, may be inserted in the homologous recombination vector for screening clones that could satisfy homologous recombination. Different systems for selection of cells that created the homologous recombination event have been described; it is worth mentioning the first described system that uses positive/negative selection vectors (Mansour et al., 1988, Capecchi, 1989).

A selection gene is a gene that enables cells that have the gene to be selected specifically for or against the presence of a corresponding selective agent. To illustrate this point, a gene with resistance to antibiotics may be used as a positive selection marker gene that enables a host cell to be positively selected in the presence of the corresponding antibiotic. The man skilled in the art will be familiar with a variety of positive and negative markers (See U.S. Pat. No. 5,627,059 for a review). This selection gene may be located inside or outside the linearised transgene. When the selection gene is located inside the transgene, in other words between the ends 5′ and 3′ of the transgene, the transgene may be present in the form of a genic entity distinct from the gene coding for at least one human polypeptide involved in the antigenic recognition and cell activation by T cells according to the invention. In this case, the selection gene is operationally bound with DNA sequences to control its expression; alternatively, the selection gene may be controlled by sequences for regulation of the expression of the said human gene. These sequences, known to an expert in the subject, correspond particularly to promoting sequences, optionally to activating sequences and to transcription termination signals. Optionally, the selection gene may form a fusion gene with the human gene. The said fusion gene is then operationally bound with DNA sequences to control the expression of the said fusion gene. According to another embodiment of the invention, the selection gene is located at the ends 5′ and 3′ of the transgene such that if a homologous recombination event occurs, the selection gene is not integrated in the cell genomic DNA; in this case, the selection gene is a negative selection gene (see U.S. Pat. No. 5,627,059 for a review).

Said positive selection gene according to the invention is preferably chosen from among genes resistant to antibiotics. Antibiotics non-exhaustively include neomycin, tetracycline, ampicillin, kanamycin, phleomycine, bleomycine, hygromycine, chloramphenicol, carbenicilline, geneticin, puromycine. The man skilled in the art will be familiar with resistance genes corresponding to these antibiotics; for example, the neomycin gene makes cells resistant to the presence of antibiotic G418 in the culture medium. The selected positive selection gene may also be the HisD gene, the corresponding selective agent being histidinol. The selected positive selection gene may also be the guanine-phosphoribosyl-transferase (GpT) gene, the corresponding selective agent being xanthine. The selected positive selection gene may also be the hypoxanthine phosphoribosyl transferase (HPRT) gene, the corresponding selective gene being hypoxanthine.

The selected said negative selection gene according to the invention is preferably the 6-thioxanthine gene or the thymidine kinase (TK) gene (Mzoz et al., 1993), genes coding for bacterial or viral toxins, for example such as the Pseudomonas exotoxin A, diphtheric toxin (DTA), choleric toxin, the Bacillus anthrox toxin, the Pertussis toxin, the Shiga Shigella toxin, the toxin related to the Shiga toxin, Escherichia coli toxins, colicine A, d-endotoxin. Note also rat cytochrom p450 and cyclophosphophamide (Wei et al., 1994), Escherichia coli (E. coli) purine nucleoside phosphorylase, and 6-methylpurine deoxyribonucleoside (Sorscher et al., 1994), cytosine deaminases (Cdase) or uracil phosphoribosyl transferase (UPRTase) that may be used with 5-fluorocytosine (5-FC).

The selection marker(s) used to be able to identify homologous recombination events may subsequently affect the gene expression and may be eliminated if necessary by the use of site specific recombinases such as Cre recombinase specific to Lox sites (Sauer, 1994; Rajewsky et al., 1996; Sauer, 1998) or FLP specific to FRT sites (Kilby et al., 1993).

Positive colonies, in other words colonies containing cells in which at least one homologous recombination event occurred, are identified by an analysis by southern blotting and/or by PCR techniques. The expression rate, in isolated cells or cells of the transgenic animal according to the invention, of the mRNA corresponding to the transgene, can also be determined by techniques including analysis by northern blotting, or in situ hybridation analysis, by RT-PCR. Animal cells or tissues expressing the transgene can also be identified using an antibody directed against the reporter protein. The positive cells can then be used to make modifications on the embryo and particularly injection of modified cells by homologous recombination into the blastocysts. For mice, blastocysts are obtained from superovulated females at 4 or 6 weeks. The cells are trypsined and the modified cells are injected into the blastocele of a blastocyst. After injection, the blastocysts are introduced into the uterine horn of pseudo-gestating females. The females are then allowed to continue their pregnancy until its termination and the resulting litters are analysed to determine the presence of mutant cells possessing the construction. The analysis of a different phenotype between the cells of the newborn embryo and the blastocyst cells or the ES cells provided means of detecting chimeric newborn. The chimeric embryos are then raised to adult age. The chimers or chimeric animals are animals in which only a sub-population of the cells has an altered genome. The chimeric animals with the modified gene or genes, are usually crossed with each other or with a wild animal in order to obtain heterozygote or homozygote progeny. The male and female heterozygotes are then crossed to generate homozygote animals. Unless mentioned otherwise, the transgenic animal according to the invention comprises stable changes to the nucleotide sequence of germ line cells.

According to another embodiment of the invention, the non-human transgenic cell according to the invention can act as nucleus donor cell in the context of a transfer of a nucleus or a nuclear transfer. A nuclear transfer means the transfer of a nucleus from a living vertebrate donor cell, an adult organism or an organism at the foetal state, into the cytoplasm of an enucleated receptor cell of the same species or a different species. The transferred nucleus is reprogrammed to direct development of cloned embryos that can then be transferred into carrier females to produce foetuses or newborn, or used to produce cells in the internal cell mass in culture. Different nuclear cloning techniques could be used; it is worth mentioning the techniques described in patent applications WO 95 17500, WO 97 07668, WO 97 07669, WO 98 30683, WO 99 01163, WO 99 37143, although this list is not exhaustive.

According to one preferred embodiment of the invention, the gene targeting according to this invention is a knock-in (K-I). The transgene or the exogenous gene or the nucleotide sequence according to the invention encoding all or at least part of a human polypeptide involved in the recognition and/or antigenic activation by T cells according to the invention, is targeted by homologous recombination in the genome of the organism. According to one preferred embodiment, the nucleotide sequence is stably integrated into the genome of the said cell by targeted insertion by homologous recombination (knock-in), at least one allele of the said animal gene, and its integration invalidates the said homologous endogenous animal gene.

According to a first embodiment of the invention, the transgene or the nucleotide sequence is deprived of gene expression regulation elements and is operationally bound to sequences for regulation of the expression of the said homologous endogenous animal gene.

According to a second embodiment of the invention, the transgene or the nucleotide sequence comprises elements for regulation of the gene expression and is operationally linked to exogenous sequences for regulation of the expression. According to one preferred embodiment, the said exogenous expression regulation sequences are regulation sequences of the expression of the said human gene encoding the human polypeptide.

The transgene comprises at least one human gene that is encoding the human polypeptide involved in the antigenic recognition and/or cell activation of T cells. Said human gene comprises either all sequences containing information for the regulated production of the corresponding RNA (transcription) or the corresponding polypeptide chain (transcription-translation). The said human gene may be a “wild” type gene with a natural polymorphism or a genetically modified DNA sequence, for example with deletions, substitutions or insertions in coding or non-coding regions. Preferably, the human gene(s) is (are) deprived of the regulation sequences necessary to direct and control their expression in one or more appropriate cell types; they are placed after homologous recombination under the control of endogenous animal sequences for regulation of the expression of the target animal endogenous gene that preferably remains active after the homologous recombination event and integration of the human gene.

Alternately, the transgene according to the invention may contain appropriate regulation sequences for directing and controlling the expression of the said human protein(s) involved in the recognition and/or antigenic activation by T cells in the cell. In this case, the transgene is integrated into the genome in a targeted or in a random manner, or is present in the cell in episomal form. In this case, the appropriate regulation sequences are sequences that can be induced by one or several proteins.

Regulation elements of the gene expression refer to all DNA sequences involved in regulation of the gene expression, in other words essentially the sequences regulating the transcription, splicing and translation. Some DNA sequences regulating the transcription that are worth mentioning include the minimum promoting sequence, upstream sequences (for example SP1 box, IRE for “interferon responsive element” etc.), activating sequences (enhancers), possibly inhibiting sequences (“silencers”), insulating sequences (“insulator”), and splicing sequences.

These expression regulation sequences are operationally linked to the human gene(s). A nucleic sequence is “operationally linked” when it is placed in a functional relation with another nucleic acid sequence. For example, a promoter or enhancer is operationally linked to a coding sequence if it affects transcription of the said coding sequence. Concerning transcription regulating sequences, “operationally linked” means that the bound DNA sequences are contiguous, and when the objective is to bind two contiguous coding regions for proteins, it means that they are in the reading phase.

The non-human transgenic cell and/or transgenic animal according to the invention is obtained by introducing at least one transgene encoding a human polypeptide involved in antigen recognition and/or cell activation by T cells, into a cell, a zygote or a young embryo of a non-human animal. Different transgenes according to the invention can also be introduced into the cell simultaneously or at different times. When the cell contains several transgenes, it can be obtained directly by simultaneous introduction of the DNA fragments necessary for homologous recombination into the said cell, using methods facilitating co-transformation of multiple DNA molecules. The cells are then selected for expected multiple recombination events using an adapted selection system. Alternately, the multi-transgenic cell may be obtained by performing homologous recombination events separately and at different times. Thus, after a first homologous recombination vector has been introduced, the cell is selected for the first homologous recombination event using an appropriate selection system; this newly transgenic cell is then transformed using a second homologous recombination vector and is then selected for the second homologous recombination event using an identical or a different selection system. Optionally, this double transgenic cell can then be transformed with a third homologous recombination vector and then selected for the third homologous recombination event using an identical or a different selection system, and so on. Alternatively, the double, triple or multi-transgenic cell according to the invention can be obtained by successive crossing of transgenic animals. For example, a double transgenic cell may be obtained by crossing two simple homozygote transgenic animals; it may be obtained by crossing and then selecting two single heterozygote transgenic animals or by crossing and selecting a single homozygote transgenic animal and a single heterozygote transgenic animal.

According to another embodiment of the invention, the cell according to the invention is characterised in that it also comprises at least one transgene comprising all or at least part of a nucleotide sequence encoding all or at least part of a human polypeptide involved in the antigenic recognition and/or the cell activation of the T cells present in said cell in episomal form, and in that the said homologous endogenous animal gene is invalidated in the said cell. Preferably, said homologous endogenous animal gene is invalidated by targeted homologous recombination (knock-out). The man skilled in the art would be capable of defining the nature and characteristics of the expression vector used to maintain the transgene in the cell according to the invention, and for its expression in episomal form.

Alternately, the cell according to the invention is characterised in that it also comprises at least one transgene comprising all or at least part of a nucleotide sequence encoding all or at least part of a human polypeptide involved in antigenic recognition and/or cell activation by T cells integrated into the genome at random; in this case, the transgene is preferably integrated into a non coding region of the genome, and is dependent on elements of the response to proteins involved in the recognition and/or antigenic activation by T cells.

According to a first embodiment of the invention, the cell according to the invention is characterised in that said nucleotide sequence(s) is (are) encoding all or part of a human class I HLA antigen and is or are inserted by targeted insertion by homologous recombination (knock-in) at the homologous animal genes coding the animal antigen(s) of the class I major histocompatibility complex (MHC I).

According to another embodiment, the cell according to the invention is characterised in that the said nucleotide sequence(s) is (are) encoding all or some of the class II HLA molecules and is or are inserted by targeted insertion by homologous recombination (knock-in) at the homologous animal gene(s) coding animal antigens of the class II major histocompatibility complex (MHC II).

According to another embodiment of the invention, the cell according to the invention is characterised in that the said nucleotide sequence(s) is (are) encoding all or some of the class I and class II HLA molecules and is (are) inserted by targeted insertion by homologous recombination (knock-in) at the homologous animal genes coding the animal antigens of the class I (MHC I) and class II (MHC II) major histocompatibility complex.

The said human class I HLA antigen is chosen from among the group composed of HLA-A2, HLA-A24, HLA-A1, HLA-A3, HLA-B7, HLA-B27, HLA-B44, HLA-B8, HLA-B35, HLA-CW7, HLA-CW3, and the said MHC I animal antigen is chosen from among H2K, H2D and H2L. The said human class II HLA antigen is chosen from among the group composed of HLA-DR4, HLA-DR1, HLA-DR11, HLA-DR7, HLA-DR2, HLA-DR3, HLA-DQ8, HLA-DQ3, HLA-DP4 and the said MHC II animal antigen is chosen from among I-A alpha, I-A beta and I-E alpha and I-E beta.

According to another embodiment, the cell according to the invention is characterised in that the said nucleotide sequence is encoding all or part of the human β2-microglobulin, and is inserted by targeted insertion by homologous recombination (knock-in) at the homologous animal gene coding β2-microglobulin.

According to another embodiment, the cell according to the invention is characterised in that the said nucleotide sequence(s) is (are) encoding all or part of at least one of the polypeptides of the human CD3 complex and is or are inserted by targeted insertion by homologous recombination (knock-in) at the homologous animal genes coding for the polypeptide(s) in the CD3 complex.

According to another embodiment, the cell according to the invention is characterised in that the said nucleotide sequence is encoding all or part of the human CD4 polypeptide and is inserted by targeted insertion by homologous recombination (knock-in) at the homologous animal gene coding for the CD4 polypeptide.

According to another embodiment, the cell according to the invention is characterised in that the said nucleotide sequence is encoding all or part of the human CD8 polypeptide and is inserted by targeted insertion by homologous recombination (knock-in) at the homologous animal gene coding for the CD8 polypeptide.

According to another embodiment, the cell according to the invention is characterised in that it comprises (a) the said nucleotide sequence encoding all or part of human β2-microglobulin, inserted by targeted insertion by homologous recombination (knock-in) at the homologous animal gene coding β2-microglobulin; and/or (b) the said nucleotide sequence coding for all or part of the human CD4 polypeptide inserted by targeted insertion by homologous recombination (knock-in) at the homologous animal gene coding for the CD4 polypeptide; and/or (c) the said nucleotide sequence coding for all or part of the human CD8 polypeptide, inserted by targeted insertion by homologous recombination (knock-in) at the homologous animal gene coding for the CD8 polypeptide. According to one preferred embodiment, only the extracellular part of the CD4 and CD8 polypeptides is humanised. Optionally, the cell according to the invention also comprises the said nucleotide sequence(s) coding for all or at least part of one of the polypeptides of the human CD3 complex, inserted by targeted insertion by homologous recombination (knock-in) at the homologous animal genes coding for the polypeptide(s) of the CD3 complex.

This invention also relates to the non-human transgenic animal comprising at least one cell according to the invention. A “transgenic animal” denotes a non-human animal, preferably a mammal chosen from among the rodents group and particularly the mouse, rat, hamster and guinea pig. The mouse is particularly appreciated because its immune system has been studied in detail. Alternatively, the transgenic animal is chosen from among bred animals and particularly from porcines, ovines, caprinae, bovines, equidae and particularly horses, and lagomorphs, particularly rabbits. The transgenic animal according to the invention can also be chosen from among primates, particularly monkeys such as the macaque, chimpanzee and the baboon.

Considering genetic polymorphisms present in the population, it may be useful if transgenic animals according to the invention and particularly transgenic mice according to the invention have different genetic pools, to facilitate the analysis or obtain a characteristic physiological or behavioural response. Thus, mice according to the invention may be selected from inbred murine lines (129Sv, 1290la, C57B16, BalB/C, DBA/2, but also in outbred lines or hybrid lines).

The transgenic animal according to the invention comprises at least one cell in which the genome comprises at least one transgene or nucleotide sequence according to the invention integrated by targeted insertion (knock-in) and optionally at least one transgene or nucleotide sequence present either in the form of an extra-chromosomal element or integrated at random in the chromosome DNA. Preferably, all transgenes according to the invention are integrated by targeted homologous recombination (knock in) into the genome of the cell according to the invention. Preferably, all animal cells and particularly its cells in the germ line are transgenic.

The transgenic animal according to the invention is characterised in that the cells in its immune system express at least one functional human HLA antigen; the cells in its immune system can also express humanised and functional co-receptor and co-stimulating molecules.

The invention is also aimed at the use of a cell and/or an animal according to the invention for screening compounds modulating the human immune response. Therefore, one purpose of the invention is to provide a process for screening compounds modulating, in other words inducing, stimulating, inhibiting or eliminating an immune response in humans, characterised in that it comprises the following steps (a) contacting a cell and/or an animal according to the invention with an immunogen responsible for initiating an immune response, (b) contacting a cell and/or an animal according to the invention with an immunogen responsible for initiating an immune response with the said compound, either simultaneously or later, (c) qualitatively and optionally quantitatively determining and evaluating of whether or not an immune response occurs, (d) then identifying the compound that selectively modulates the immune response.

According to one embodiment, determining and/or evaluating said immune response is realised using a technique selected from (a) determination of the production of soluble factors such as chemokines and cytokines, (b) determination of the presence of receptors on the cell surface, (c) determination of cell proliferation, (d) determination of T cell effector functions (CTL, Helper, etc.), (e) determination of the production of antibodies by B cells.

Alternately, said determining and/or evaluating of said immune response is realised by measuring the expression ratio of a reporter gene. For the purposes of this invention, a reporter gene means a gene that enables cells containing this gene to be detected specifically following expression of this gene, in other words to be distinguished from other cells that do not contain this marker gene. The said reporter gene according to the invention is coding for a reporter protein preferably chosen from among the group composed of self-fluorescent proteins such as the green fluorescence protein (GFP), the enhanced green fluorescence protein (EGFP), the yellow fluorescence protein (YFP), the cyan fluorescence protein (CFP), the red fluorescence protein (RFP), and variants of these fluorescence proteins obtained by mutagenesis to generate a different colour fluorescence. The said reporter gene is also coding for any enzyme that can be detected by fluorescence, phosphorescence, or visible by a histochemical process on living cells or any other cell analysis methods, or by microscopy. Non-exhaustively, it is worth mentioning β-galactosidase (β-GAL), β-glucoronidase (β-GUS), alkaline phosphatase and particularly placental alkaline phosphatase (PLAP), alcohol dehydrogenase, and particularly alcoholic drosophile (ADH) dehydrogenase, luciferase, and particularly “Firefly Luciferase”, chloramphenicol-acetyl-transferase (CAT), and the growth hormone (GH).

Finally, the invention also relates to the use of a composition comprising a compound modulating the immune response and a pharmaceutically acceptable vehicle for a medicine for preventive and/or curative treatment for a man or an animal requiring such a treatment, characterised in that the aptitude of the said compound to modulate, in other words to inhibit, activate, annihilate the immune response selectively is determined by (a) contacting a cell and/or an animal according to the invention with an immunogen responsible for initiating an immune response, (b) contacting a cell and/or an animal according to the invention with an immunogen responsible for initiating an immune response with the said compound either simultaneously or later, (c) qualitatively and optionally quantitatively determining and evaluating whether or not an immune response occurs, (d) then identifying the compound that selectively modulates the immune response.

For the purpose of the invention, an antigen refers to a compound capable of initiating an immune response and/or being recognised by an antibody or a T lymphocyte. An immunogen refers to a compound capable of initiating an immune response. Antigens that react with T cell receptors or with any other types of receptors expressed on cells involved in the initiation and development of an innate or specific immune response, include allergens, mitogens, pathogenic agents or one of their constituents, with a viral, bacterial, parasite, fungal, mycoplasmic origin, vaccines, and vaccine compositions, additives, medicines, chemical compounds or chemical agents. A specific antigen can be brought into contact with a cell or an animal according to the invention by various methods for example such as a classical infection by a pathogenic microorganism, or through a biological delivery vector (mosquito, tick, bacteria, virus and parasites or a recombining commensal agent, bare DNA, etc.), by inhalation, in aerosol, through food. Experimentally, the immunogen may be brought into contact with the animal by administration by systemic pathways, particularly an intravenous pathway, intramuscular pathway, intradermic pathway, skin contact or orally.

The compounds obtained by screening processes according to the invention and that induce an immune response in humans form excellent vaccines. These compounds thus identified may for example be minimal epitope vaccines for viral diseases such as the human Acquired ImmunoDeficiency Syndrome (AIDS) provoked by an infection through HIV (human immunodeficiency virus), hepatitis B, hepatitis C for bacterial diseases such as tuberculosis, or from parasite sources such as malaria.

The compound obtained by the screening process according to the invention or the composition according to the invention can be used not only for a preventive treatment, but also for a remedial treatment for a number of pathologies for which there is a dysfunction of antigenic recognition and/or cell activation by T cells. This is the case particularly in the context of a bacterial, viral, fungal or parasite infection or for the initial development of a cancer and auto-immune diseases. Auto-immune diseases non exhaustively include uveitis, Bechet's disease, Sarcoidosis, Sjorgren's syndrome, rhumatoid polyarthritis, juvenile polyarthritis, Fiessinger-Leroy-Reiter syndrome, gout, osteorarthritis, disseminated acute erythematous lupus, polymyositis, myocarditis, primitive biliary cirrhosis, Crohn's disease, ulcerous colitis, multiple sclerosis and other demyelinating diseases, aplasic anemia, essential thrombocytopenic purpura, multiple myeloma, and lymphoma with B lymphocytes, Simmonds' panhypopituitarism, Basedow-Graves' disease and Graves' opthalmopathy, subacute thyroiditis and Hashimoto's disease, Addison's disease, and insulino-dependent diabetes mellitus (type 1).

A pharmaceutically acceptable vehicle refers to any type of vehicle normally used in preparation of pharmaceutical and vaccine compositions, in other words a diluent namely a synthetic or biological vector, a suspension agent such as an isotonic or buffered saline solution. Preferably, these compounds will be administered systemically, particularly by an intravenous pathway, or intramuscular pathway, intradermic pathway or oral pathway. Their methods of administration, posologies and optimum galenic forms may be determined using criteria usually determined in setting up a treatment adapted to a patient, for example such as the age or the body weight of the patient, the gravity of his general condition, tolerance to the treatment and observed secondary effects, etc. When the agent is a polypeptide, an antagonist, a ligand, a polynucleotide, for example an antisense composition, a vector, for example such as an antisense vector, it can be introduced in tissues or host cells in a number of ways including viral infection, micro-injection or fusion of vesicles. Injection by jet is also possible for intramuscular administration.

The invention relates to the use of a cell or an animal according to the invention for experimental research purposes for analysis, study and modelling of molecular, biological, biochemical, physiological and/or physiopathological mechanisms of the immune response in humans and particularly antigenic recognition and/or cell activation by T cells. The complete animal or cells derived from the said animal may be used, depending on the type of research to be developed. These cells may be either freshly isolated from the animal or they may be immortalised in culture, either by multiplying passages or by transforming cells by viruses such as the SV40 virus or the Epstein-Bahr virus. Thus, cells and animals according to the invention are particularly useful to study molecular bases necessary for setting up and developing autoimmune diseases, allergenic phenomena or inflammatory phenomena, and graft rejections.

The invention relates to the use of a cell or an animal according to the invention for screening therapeutically active biological or chemical compounds and particularly compounds modulating the human immune response.

The invention also relates to the use of a cell genetically modified ex vivo according to the invention for preparation of a cell and/or tissue graft for preventive or curative treatment of a human or animal necessitating such a treatment, characterised in that when an allogeneic host is transplanted with the said cell, the cell is less strongly rejected or better tolerated than a cell that was not genetically modified by the immune system of the said host. Preferably, the said cell is a mouse, pig, bovine or primate cell. Preferably, it is pig cell. This type of cell could form universal donor cells and/or donor cells personalised by the nature of the expressed human HLA molecules. Particularly interesting cells include Langerhans cells, subrenal medulla cells that can secrete dopamine, osteoblasts, osteoclasts, epithelial cells, endothelial cells, T lymphocytes, neurons, glial cells, ganglion cells, renal cells, retina cells, embryonic stem cells, hepatic cells, bone marrow cells and myoblasts. The said cell also expresses at least one protein intended for preventive and remedial treatment of a human or animal requiring such a treatment, the said protein preferably being selected from the group composed of cytokines, interleukins, chemokines, growth factors, hormones, antibodies. Thus for the treatment of cancer, it may be useful to graft cells according to the invention expressing interleukin 2 (IL2) or GM-CSF (granulocytes-macrophages colonies stimulating factor), to a patient suffering from cancer. Thus, it may be interesting to graft cells according to the invention expressing insulin for the treatment of diabetes.

Other characteristics and advantages of the invention will become clear after reading the description with the examples given below.

### EXAMPLES

Equipment and Methods

Vectors

The gene coding for beta 2 microglobulin in the mouse is composed of 4 exons, the exon 2 coding for almost the entire protein. It is humanised by knock-in of the second exon coding for the human protein, replacing the second murine exon.

The homologous recombination vector corresponds to a fragment of genomic DNA at the murine gene of the beta 2 microglobulin in which the exon 2 is replaced by its human homologous exon by enzymatic digestion at intron sites.

The CD8 molecule is a heterodimer formed from an alpha sub-unit and a beta sub-unit. The two genes coding for these proteins are located on a 60 kb region. This proximity obliges the inventors to necessarily do a knock-in of these genes on the same clone of ES cells and to verify that the two homologous recombinations took place on the same chromosome by FISH (Fluorescent In Situ Hybridization) with probes specific to each construction or by any other discriminating methods (for example chromosome segregation).

The two CD8 alpha and CDB beta genes are invalidated by targeted insertion in the first exon coding for a chimeric cDNA molecule comprising the human extracytoplasmic part associated with a cDNA sequence coding for transmembrane and intracytoplasmic parts of the murine molecule. The two homologous recombinations are done at the same time by co-electroporation of the two vectors, to avoid two successive homologous recombination steps.

For all these vectors, selection cassettes are flanked by site-specific recombinases so that they can be eliminated when the homologous recombination event has been selected.

H2-K is invalidated by deleting exons 1 and 2 for murine genes coding for class I MHC molecules in mice. The H2-D gene is invalidated by insertion of a selection cassette flanked by site-specific recombinases Cre so as to make an exchange. One or several chosen HLA genes are inserted in the H2-D locus by simple exchange of the cassette containing human cDNA. The inventors had initially introduced the cDNA from the HLA-A1 molecule.

Culture, Electroporation and Selection of Embryonic Stem Cells.

The ES genetic pool cells (129Sv/J or C57BL/6J) are cultivated on feeder cell layers (Mouse Embryonic Fibroblasts (MEF)) as described above (Fraîchard et al., 1997).

The ES cells are trypsined, washed and resuspended at a concentration of 6.25×10⁶ ES/ml in a culture medium without serum and are electroporated in the presence of 25 to 50 μg/ml of linearised homology vector. A voltage of 260 V associated with a 500 μF capacitance is optimum for a 4 mm thick electroporation chamber.

1×10⁶ to 5×10⁶ electroporated ES cells are then seeded on irradiated Neo resistant MEFs. 36 hours after the electroporated ES cells are put into culture, selection of resistant clones begins by adding geneticin (G418 at 250 μg/ml) into the culture medium.

For co-electroporation, an equimolar mix of two homologous recombination vectors is electroporated under the same conditions.

Analysis of Resistant Antibiotic Clones by Screening by PCR and Southern Blot

ES cell clones visible at 10 to 12 days of culture in the presence of G418 are sampled.

Three quarters of the remaining cells are put into culture on feeder cell layers in 96-well plates. The remaining quarter is handled in 96-well plates, so that 80 clones can be analysed simultaneously. ES cells are resuspended by adding 10 μl of sterile H20. A temperature shock is applied to burst the cells (2 minutes at 65° C.), and 4 μl is then used for the PCR reaction. Recombining clones isolated by PCR are confirmed by Southern blot.

Production of Chimeric Mice by Injection of ES Cells into Blastocysts

Blastocysts are isolated from C57BL/6J donor females (Charles River Iffa Credo) 3.5 days after fertilization. Blastocysts are retrieved by rinsing uterine horn with 1 ml of the M2 medium. Some blastocysts are deposited in the injection chamber, in a drop of M2 covered with mineral oil. 3 to 5 ES cells are injected in the blastocoel. 4 hours after injection, 5 to 9 blastocysts are reimplanted in each uterine horn of pseudogestating females mated 2.5 days earlier with a vasectomised male.

The ES genetic pool cells 129Sv/J and all mice derived from these ES cells carry markers characteristic of the strain, in other words homozygote for the A/A agouti locus giving an agouti colour hair coat. The contribution of ES cells to the development of the host embryo (C57BL/6J) (not agouti) can be quickly evaluated at the hair coat. If the injected ES cells participated in the embryonic development, the mice obtained have an agouti and black chimeric hair coat very easily identifiable from all-black young originating from host embryos not colonised by ES cells. According to the same principle, recombining ES C57BL/6J cells (black) are injected into blastocysts from the BALB/C genetic pool (albino).

Generation of Heterozygote Animals

Males with a high rate of chimerism are mated with C57BL/6J females.

Chimers obtained by injection of ES C57BL/6 cells are also mated with C57BL/6J females. In this case, the entire first generation is screened by PCR for the homologous recombination event. Animals found to be positive heterozygote by PCR are systematically confirmed by Southern blot.

DNA for genotyping of progeny is obtained by biopsies on mouse tails.

Generation of Homozygote Animals

Heterozygote males and females are mated, and the litters are analysed for the presence of two recombining alleles. As expected, a quarter of the progeny is homozygote. These animals then represent a new line of transgenic mice.

Transgenic mice expressing human polypeptides involved in recognition and/or antigenic activation by T cells will be produced independently. Homozygotes and/or heterozygotes for each transgenic type will then be crossed and the progeny will be tested to select animals expressing both transgenic types.

The genetic pool of transgenic animals could also be changed by successive crossings with animals from a genetic pool different from the pool used initially.

### REFERENCES

Barzaga-Gilbert et al. (1992) J. Exp. Med. 175: 1707-1715.
Bradding et al. (1992) J. Exp Med. 176: 1381.
Capecchi (1989) Science 244: 1288.
Costa et al. (2000) J. Immunol. 164: 3581
Dalton et al. (1993) Science 259: 1739.
Del Prete et al. (1991) J. Clin Invest. 88: 346
Farrar (1993) Annu. Rev. Immunol. 11: 571
Firestein et al (1989) J. Imm. 143: 518
Fraichard et al. (1997) EMBO J 14: 4412-20.
Gordon et al. (1989) Transgenic Animals, Intl. Rev. Cytol. 115: 171
Gray (1983) Proc. Natl. Acad. Sci. USA 80: 5842.
Gross and Paul (1993) J. Immunol. 150: 2112.
Groux et al. (1997) Nature 339: 152.
Hu-Li J. et al. (2001) Immunity 14: 1-11.
Ito et al. (1996) J. Exp. Med. 183: 2635-2644.
Keon et al. (1990) Methods and Enzymology 185: 527.
Kilby et al. (1993) TIG 9: 413.
Kopf et al. (1993) Nature 362: 245
Kuhn and Muller (1991) Science 254: 707.
Lavitrano et al. (1989) Cell 57: 717.
Lee et al. (1986) Proc. Natl. Acad. Sci. USA 83: 2061
Lo-Weber et al. (1997) Immunobiology 198: 170.
Liu et al. (2000) Anal. Biochem. 280: 20.
Lo (1983) Mol. Cell. Biol. 3: 1803
Mansour et al. (1989) Ann. Res. Genet. 23: 199.
Metwali et al. (1996) J. Immunol. 157: 4546.
Mosmann et al. (1986) J. Immunol. 136: 2348.
Mosmann et al. (1989) Ann. Rev. Immunol. 7: 145
Mosmann (1996) Immunol. Today 17: 138.
Mountford (1995) Trends in Genetics 11: 179.
Mzoz et al. (1993) Human Gene Ther. 4: 589.
Neddleman et al. (1970) J. Mol. Biol. 48: 443.
Noben-Trauth et al. (1996) Transgenic Res. 5: 487.
Otsuka et al. (1987) Nucleic Acids res. 15: 333.
Ouyang et al. (2000) Immunity 12: 27.
Pascolo et al (1997) J. Exp. Med. 185: 2043-2051.
Pearson et al (1988) Proc. Natl. Acad. Sci. USA 85: 2444
Ranganath et al. (1998) J. Immunol. 161: 3822.
Robinson et al (1993) J. Imm. 143: 518.
Romagnani (1999) Inflamm. Bowel Dis. 5: 285.
Sambrook et al. (1989) Molecular cloning: a laboratory manual second edition—Cold Spring Harbor Laboratory Press. Cold Spring Harbor, N.Y. USA.
Sauer (1994) Current opinion in Biotechnology 5: 521.
Sauer B. (1998) Methods 14: 81-392.
Sideras et al. (1987) Adv. Exp. Med. Biol. 213: 227.
Smith and Waterman (1981) Ad. App. Math. 2: 482.
Sorscher et al. (1994) Gene Therapy 1: 223-238.
Swain et al. (1990) J. Immunol. 145: 3796.
Szabo et al. (2000) Cell 100: 655.
Thompson et al (1989) Cell 56: 313.
Van der putten et al. (1985) Proc. Natl. Acad. Sci. USA 82: 6148.
Wei et al. (1994) Human Gene Ther. 5: 969-978.
Wiernenga et al. (1990) J. Imm. 144: 4651.
Yamamura et al. (1991) Science 254: 77.
Zheng (1997) Cell 89: 587.
Zhu and grace (1999) Cytometry 37: 51.

1. An isolated animal cell comprising at least one transgene comprising at least one nucleotide sequence encoding all or at least part of one human polypeptide involved in antigenic recognition and/or cell activation of T cells, wherein said cell, or a progeny of said cell, expresses all or at least some of said human polypeptide(s), and wherein said nucleotide sequence is integrated into the genome of said cell in a stable manner by a targeted insertion by homologous recombination (Knock-in) at least one allele of said endogenous animal gene, the integration of said sequence invalidating said homologous endogenous animal gene, wherein the animal is a murine; and
wherein said animal further comprises a nucleotide sequence which encodes all or part of a human class II HLA antigen and is (are) inserted by targeted insertion by homologous recombination (Knock-In) at the homologous animal gene(s) encoding the animal antigens of the class II major histocompatibility complex (MHC II); and
wherein said nucleotide sequence encodes all or part of the human CD4 polypeptide and is inserted by targeted insertion by homologous recombination (knock-in) at the homologous animal gene encoding the CD4 polypeptide.

2-3. (canceled)

4. The cell according to claim 1, wherein the said nucleotide sequence is operationally linked to expression regulation sequences of said homologous endogenous animal gene.

5. The cell according to claim 1, wherein said nucleotide sequence is operationally linked to exogenous expression regulation sequences.

6. The cell according to claim 5, wherein said exogenous expression regulation sequences are the regulation sequences for expressing the human gene encoding the human polypeptide.

7. The cell according to claim 1, which further includes at least one transgene comprising at least all or part of a nucleotide sequence encoding at least all or part of a human polypeptide involved in antigenic recognition and/or cell activation of T cells present in said cell in episomal form, and wherein said homologous endogenous animal gene is invalidated in said cell.

8. The cell according to claim 7, wherein said homologous endogenous animal gene is invalidated by targeted homologous recombination (Knock-Out).

9. The cell according to claim 1, wherein said nucleotide sequence(s) encodes all or part of a human class I HLA antigen and is (are) inserted by targeted insertion by homologous recombination (Knock-In) at the homologous animal gene(s) encoding the animal antigens of the class I major histocompatibility complex (MHC I).

10. The cell according to claim 1, wherein said nucleotide sequence(s) encode(s) all of class II HLA molecules and is (are) inserted by targeted insertion by homologous recombination (Knock-In) at the homologous animal gene(s) encoding the animal antigens of the class II major histocompatibility complex (MHC II).

11. The cell according to claim 40, wherein said nucleotide sequence(s) encode(s) all or part of class I and class II HLA molecules and is (are) inserted by targeted insertion by homologous recombination (Knock-In) at the homologous animal gene(s) encoding the animal antigen(s) of the class I major histocompatibility complex (MHC I) and class II major histocompatibility complex (MHC II).

12. The cell according to claim 11, wherein said human class I HLA antigen is selected from the group consisting of HLA-A2, HLA-A24, HLA-A1, HLA-A3, HLA-B7, HLA-B27, HLA-B44, HLA-B8, HLA-B35, HLA-CW7, HLA-CW3 and wherein said MHC I animal antigen is selected from the group consisting of H2K, H2D and H2L.

13. The cell according to claim 1 or 11, wherein the said human class II HLA antigen is selected from the group consisting of HLA-DR4, HLA-DR1, HLA-DR11, HLA-DR7, HLA-DR2, HLA-DR3, HLA-DQ8, HLA-DQ3, HLA-DP4, and wherein said MHC II animal antigen is selected from the group consisting of I-A alpha, I-A beta and I-E alpha and I-E beta.

14. (canceled)

15. The cell according to claim 1, which further includes sequence(s) that encode(s) all or part of at least one of the polypeptides of the human CD3 complex and is or are inserted by targeted insertion by homologous recombination (knock-in) at the homologous animal gene(s) encoding the polypeptide(s) in the CD3 complex.

16-17. (canceled)

18. The cell according to claim 9, which further comprises:
a) said nucleotide sequence encoding all or part of human β2-microglobulin, inserted by targeted insertion by homologous recombination (knock-in) at the homologous animal gene encoding β2-microglobulin; and/or
b) said nucleotide sequence encoding for all or part of the human CD3 polypeptide inserted by targeted insertion by homologous recombination (knock-in) at the homologous animal gene encoding the CD3 polypeptide; and/or
c) said nucleotide sequence encoding all or part of the human CD8 polypeptide, inserted by targeted insertion by homologous recombination (knock-in) at the homologous animal gene coding for the CD8 polypeptide.

19-22. (canceled)

23. The cell according to claim 1, wherein said cell is selected from the cells of the immune system, professional and non-professional antigen presenting cells, hematopoietic stem cells and embryonic stem cells.

24. The cell according to claim 23, wherein said cell in the immune system is selected from all types of mature and immature T lymphocytes, thymocytes, dendritic cells, intra-epithelial lymphocytes, NK cells, B cells, monocytes, professional and non-professional antigen presenting cells.

25. The stem cell according to claim 23, wherein said stem cell is subsequently differentiated as a cell selected from the immune system cells.

26. A transgenic murine animal, comprising at least one cell according to claim 1.

27. The animal according to claim 26, which is selected from the group consisting of mouse, rat, hamster, guinea pig.

28. The animal according to claim 27, wherein the animal is a mouse.

29. The animal according to claim 26, wherein the cells of its immune system express at least one functional human HLA antigen.

30. The animal according to claim 29, wherein the cells of its immune system also express humanised and functional co-receptor and co-stimulating molecules.

31. A process for screening a compound modulating an immune response in humans, which comprises the following steps:
a) contacting a cell according to claim 1, with an immunogen responsible for initiating an immune response;
b) contacting said cell with an immunogen responsible for initiating an immune response, and, either simultaneously or later, with said compound;
c) qualitatively and optionally quantitatively determining and evaluating whether or not an immune response occurs;
d) then identifying the compound that selectively induces the immune response.

32. The process according to claim 31, wherein determining and/or evaluating said immune response is realised using a technique selected from the group consisting of:
a) determination of the production of soluble factors such as chemokines and cytokines,
b) determination of the presence of receptors on the cell surface,
c) determination of cell proliferation,
d) determination of T cell effector functions and,
e) determination of the production of antibodies by B cells.

33. The process according to claim 31, wherein determining and/or evaluating said immune response is realised by measuring the expression ratio of a reporter gene.

34. A method for analysis, study and modelling of molecular, biological, biochemical, physiological and/or physiopathological mechanisms of the immune response in humans comprising using the cell according to claim 1.

35. A method for screening compounds modulating the human immune response comprising using the cell according to claim 1.

36. A method for preparation of a cell and/or tissue graft for preventive or curative treatment of a human or animal necessitating such a treatment, wherein when an allogeneic host is transplanted with said cell, this cell is less strongly rejected or better tolerated than a cell that was not genetically modified, by the immune system of said host.

37. (canceled)

38. The cell according to claim 1 which also expresses at least one protein for preventive and curative treatment of a human or animal requiring such a treatment, the said protein being preferably selected from the group composed of cytokines, interleukins, chemokines, growth factors, hormones antibodies.

39. The cell according to claim 1 which also expresses at least one protein for preventive and curative treatment of a human or animal requiring such a treatment, the said protein being preferably selected from the group composed of cytokines, interleukins, chemokines, growth factors, hormones and antibodies.

40. The cell according to claim 1, wherein the murine is a mouse.

41. The cell according to claim 1, wherein the murine cell further comprises a transgene encoding human HLA-I.

41. The cell according to claim 39, wherein said animal further comprises a nucleotide sequence which encodes all or part of the human β2-microglobulin, and is inserted by targeted insertion by homologous recombination (knock-in) at the homologous animal gene encoding β2-microglobulin.

42. The cell according to claim 1, wherein said animal further comprises a nucleotide sequence which encodes all or part of the human CD8 polypeptide and is inserted by targeted insertion by homologous recombination (knock-in) at the homologous animal gene encoding the CD8 polypeptide.
